(19) United States
(12) Patent Application Publication
(10) Pub. No.: US 2006/0063702 A1
(43) Pub. Date: Mar. 23, 2006

(54) Neuromedin U 2 receptor agonists and uses thereof

(76) Inventor: Yinghe Hu, San Diego, CA (US)

Correspondence Address: LAW OFFICES OF ALBERT WAI-KIT CHAN, LLC, WORLD PLAZA, SUITE 604, 141-07 20TH AVENUE, WHITESTONE, NY, 11357, US

(21) Appl. No.: 11/222,927

(22) Filed: Sep. 8, 2005

(30) Foreign Application Priority Data
Sep. 8, 2004 (CN) 200410054396.4

(51) Int. Cl. A61K 38/17; C40B 40/10

(52) U.S. Cl. 514/2

(57) ABSTRACT

This invention provides neuromedin U 2 receptor (NMU-2R) agonists. This invention further provides a method for screening and identification of said agonist. This invention also provides the core structure for NMU-2R agonists. This invention provides that the agonist can effectively reduced body weight of high fat diet induced obese rats and normal C57BL/6 mice. In addition, the compound increased the insulin sensitivity and decreased blood glucose level of obese rats. Specifically, this invention provides that Rutin and the related compounds could be developed into effective therapeutic drugs for obesity and non-insulin dependent diabetes mellitus (NIDDM).

This invention claims priority of Chinese Application No. 200410054396.4 filed Sep. 8, 2004, the content of which is incorporated into this application by reference.

Throughout this application various references are referred to within parenthesis. Disclosures of these publications in their entireties are hereby incorporated by reference into this application to more fully describe the state of the art to which this invention pertains. Full bibliographic citation for these references may be found at the end of this application, preceding the claims.

### BACKGROUND OF THE INVENTION

Neuromedin U (NuM), a peptide hormone, is widely distributed in the gut and central nervous system^{[1-4]}. It has potent activities in many different physiological processes, including stimulation of muscle activity, regulation of blood pressure, alteration of ion transport in the gut, control of local blood flow and regulation of adrenocortical function^{[1]}. In the brain, NmU may play important roles in the circadian oscillator and food intake^{[5]}. Two G-protein coupled receptors (GPCRs) have been identified as NmU receptors. NmU 1 receptor is mainly distributed in peripheral tissues, particularly the small intestines and stomach^{[5,6]} and plays essential roles in regulating secretory function and affecting mucosal function^{[5]}. NmU 2 receptor (NMU-2R), however, is exclusively expressed in the brain^{[5,7}]. The physiological function of NMU-2R remains to be elucidated. Intracerebroventricular (ICV) administration of NmU significantly decreased food intake^{[5]}. Central effects of NmU play an important role in feeding behavior and energy homeostasis. More specifically, the effect of NmU has been mapped to particular hypothalamic nuclei. Injection of NmU into either the paraventricular nucleus (PVN) or arcuate nucleus decreases food intake in rats^{[8]}. Since NMU-2R was highly expressed in hypothalamus, it has been suggested that the central effects of NmU could be mediated through NMU-2R. Further functional studies of the receptor were necessary to validate the possibility.

Chemical genetic approach is a powerful tool to study the physiological function of GPCRs. A number of studies have demonstrated that small molecule agonists could be used to study the physiological functions of peptide receptors in the brain^{[9,10]}.

In this disclosure, the identification of several structurally related small molecule agonists for NMU-2R from a natural product library were described. Pharmacological characterization of the compounds demonstrated that they specifically activated NMU-2R. Rutin was the selected compound for detailed in vivo studies to explore the physiological function of NMU-2R. The results showed that administration of Rutin, either through oral or intraperitoneal (i.p.), significantly reduced body weight of high fat diets induced obese rats and normal mice. Furthermore, Rutin was found to increase insulin sensitivity and decrease blood glucose level after administration in obese rats.

### SUMMARY OF THE INVENTION

This invention provides a composition comprising an amount of Rutin or its derivative effective in activation of neuromedin U2 receptor. As used herein, Rutin is defined as a member of bioflavonoids, a large group of phenolic secondary metabolites of plants. As used herein, derivatives of Rutin are other natural compounds that have similar structure and activity relationship (SAR) as Rutin. In an embodiment, the Rutin is isolated from a natural product library.

In another embodiment, Rutin or its derivative is a neuromedin U2 receptor ligand. In a further embodiment, Rutin or its derivative is an agonist for the neuromedin U2 receptor.

This invention also provides a composition comprising an amount of Rutin or its derivative effective in activation of neuromedin U2 receptor to increase the insulin sensitivity. This invention provides a composition comprising an amount of Rutin or its derivative effective in activation of neuromedin U2 receptor to decrease the blood glucose level. This invention provides a composition comprising an amount of Rutin or its derivative effective in activation of neuromedin U2 receptor to suppress food intake and regulate the energy homeostasis. This invention provides a composition comprising an amount of Rutin or its derivative effective in activation of neuromedin U2 receptor to reduce body weight. This invention provides a composition comprising an amount of Rutin or its derivative effective in activation of neuromedin U2 receptor to reduce blood-fat level and concentration of fatty acids in blood. This invention provides a composition comprising an amount of Rutin or its derivatives effective in activation of neuromedin U2 receptor to treat obesity and non-insulin dependent diabetes mellitus. This invention provides a composition comprising an amount of Rutin or its derivatives effective in activation of neuromedin U2 receptor to decreases the level of total cholesterol, triglyceride, high-density lipoprotein cholesterol and lipoprotein cholesterol in blood.

This invention also provides a composition comprising an effective amount of a neuromedin U2 receptor agonist and a suitable carrier.

In an embodiment, the agonist is not previously known. This invention further provides this agonist. Regardless of whether the agonist is known or unknown, this invention provides a pharmaceutical composition comprising an effective amount of a neuromedium U2 receptor and a pharmaceutically acceptable carrier.

This invention provides a method of treating insulin-dependent diabetes mellitus comprising administering to a diabetes mellitus subject or subject at risk of becoming diabetic an effective amount of Rutin or its derivative. This invention provides a method for increasing the insulin sensitivity of a subject comprising administering to said subject an amount of Rutin or its derivative effective to increase the insulin sensitivity. This invention provides a method for decreasing blood glucose of a subject comprising administering to the subject an amount of Rutin or its derivative effective to decreases the blood glucose level. This invention provides a method for suppressing food intake and regulating energy homeostasis in a subject comprising administering to said subject an amount of Rutin or its derivative effective to suppress food intake and regulating energy. This invention provides a method for reducing the body weight of a subject comprising administering to said subject an amount of Rutin or its derivative to reduce body weight. This invention provides a method for treating obesity in a subject comprising administering to the said subject an effective amount of Rutin or its derivative. This invention provides a method for decreasing total cholesterol, triglyceride, high-density lipoprotein cholesterol and lipoprotein cholesterol in blood of a subject comprising administering an effectively amount of Rutin or its derivative.

The above described subject includes human and other animals. In an embodiment, it is a mammal.

This invention provides the above method, the Rutin or its derivative is administered orally, intravenously or intraperitoneally. Rutin or its derivative may be used with other agents. The dosage is about 1 milligram (mg) to 1 gram (g) per kilogram of the subject used.

Other neuromedin U2 receptor agonists may be used as described above.

This invention further provides a screening method for said agonist wherein the NMU-2R receptor and a reporter gene are introduced to a cell line in such a way that when an exogenous compound can activate the receptor which will lead to the reporter gene expression.

This invention also provides the agonists resulted from this screening method. Finally, different uses of the resulted agonists are decribed.

### BRIEF DESCRIPTION OF FIGURES

FIG. 1. The luciferase activities of HEK-293/NMU2R/3MCS/Luci cells stimulated with foskolin and NMU.

FIG. 2. The screening result of natural compounds.

FIG. 3. The luciferase activity of the raw extract of Pericarpium Citri Reticulatae stimulating different cells. NMU2R: HEK-293/NMU2R/3MCS/Luci cells, MC4R: HEK-293/MC4R/3MCS/Luci cells, M1R: HEK-293/M1R/3MCS/Luci cells, 3MCS: HEK-293/3MCS/Luci cells, F: forskolin (5 pmol/L), PCR: the raw extract of Pericarpium Citri Reticulatae, α-MSH: α-melanocyte stimulating hormone, Ach: acetylcholine. The concentrations (⅕ and ½) are fold-concentration of raw extracts.

FIG. 4. The luciferase activity of Icariin in Herba Epimedii and the TLC result. A) The luciferase activities of Icariin stimulating different cells. B) The TLC result of Herba Epimedii extract: NMU2R: HEK-293/NMU2R/3MCS/Luci cells, MC4R: HEK-293/MC4R/3MCS/Luci cells, M1R: HEK-293/M1R/3MCS/Luci cells, 3MCS: HEK-293/3MCS/Luci cells, F: forskolin (5 μmol/L), SSIc: standard sample of Icariin, a flavonoid glycoside in Herba Epimedii, acHE: the active compound in herba epimedii, HE: the raw extract of Herba Epimedii, α-MSH: α-melanocyte stimulating hormone, Ach: acetylcholine. The TLC was on silica gel G plate. The combined solutions of AcOEt/methanol/H₂O (100:17:13) and the upper layer of methylbenzene/AcOEt/methanoic-acid/H₂O (20:10:1) were respectively used to sweep up the sample dot on the silica plate. Then the plate was sprayed onto 3% of aluminium trichloride alcohol solution, and taken visual inspection under ultraviolet lamp (365 nm).

FIG. 5. The structures and activities of some flavonoid glycosides. The serials numbers, names and activities of some flavonoid glycosides are indicated under the structures in the figure.

FIG. 6. The based structure of the hNM2R agonist. They are flavonoid glycosides.

FIG. 7. The food intakes changes of the femal C57BL/6 mice during administration of Rutin. The femal C57BL/6 mice were administrated with two different doses of Rutin every day (30 mg/kg and 100 mg/kg)** indicates p<0.05, and indicates p<0.01 in t-test.

FIG. 8: The body weight changes every day of the femal C57BL/6 mice during 5 weeks of successive administration with Rutin and after administration stopped. The femal C57BL/6 mice had been administrated with two different doses of Rutin every day (30 mg/kg and 100 mg/kg) for 5 weeks, then, the administration was stopped. ** indicates p<0.05, and indicates p<0.01 in t-test.

FIG. 9. The fat index of the femal C57BL/6 mice after administered with Rutin. The femal C57BL/6 mice were administrated with two different doses of Rutin every day (30 mg/kg and 100 mg/kg)** indicates p<0.05 in t test.

FIG. 10. The fasting serum glucose, fasting insulin and insulin sensitivity index of the obese Wistar rats model administered with Rutin. FSG: fasting serum glucose; FI: fasting insulin; SIS: insulin sensitivity index. ** indicates p<0.01, and * indicates p<0.05 contrasted with model control groups in t-test. ## indicates p<0.01, and # indicates p<0.05 contrasted with normal control groups in t-test.

FIG. 11. The blood-fat biochemical indicator of the obese Wistar rats model administered with Rutin. TC: Total cholesterol, TG: triglyceride, HDLC: high-density lipoprotein cholesterol, LDLC: low density lipoprotein cholesterol. ** indicates p<0.01, and * indicates p<0.05 contrasted with model control groups in t-test. ## indicates p<0.01, and # indicates p<0.05 contrasted with normal control groups in t-test.

FIG. 12. The fat growth rate, fat ratio and free fatty acid of the obese Wistar rats model administered with Rutin. FFA: free fatty acid. * indicates p<0.05 contrasted with model control groups in t-test. ## indicates p<0.01 contrasted with normal control groups in t-test.

FIG. 13. The glucose infusion rate (GIR) of the obese Wistar rats model administered with Rutin. SS glucose: steady state glucose; ## indicates p<0.01 contrasted with normal control groups in t-test.

### DETAILED DESCRIPTION OF THE INVENTION

This invention provides a composition comprising an amount of neuromedin U2 receptor agonist or its derivative effective in activation of neuromedin U2 receptor. As used herein, derivatives of the agonist are molecules or compounds which have similar structure and/or activity as the agonist.

As used herein, Rutin is defined as a member of bioflavonoids, a large group of phenolic secondary metabolites of plants. As used herein, derivatives of Rutin are other natural compounds that have similar structure and/or activity relationship (SAR) as Rutin.

In an embodiment, the Rutin is isolated from a natural product library.

In another embodiment, Rutin or its derivative is a neuromedin U2 receptor ligand. In a further embodiment, Rutin or its derivative is an agonist for the neuromedin U2 receptor.

This invention also provides a composition comprising an amount of Rutin or its derivative effective in activation of neuromedin U2 receptor to increase the insulin sensitivity.

This invention provides a composition comprising an amount of Rutin or its derivative effective in activation of neuromedin U2 receptor to decrease the blood glucose level.

This invention provides a composition comprising an amount of Rutin or its derivative effective in activation of neuromedin U2 receptor to suppress food intake and regulate the energy homeostasis.

This invention provides a composition comprising an amount of Rutin or its derivative effective in activation of neuromedin U2 receptor to reduce body weight.

This invention provides a composition comprising an amount of Rutin or its derivative effective in activation of neuromedin U2 receptor to reduce blood-fat level and concentration of fatty acids in blood.

This invention provides a composition comprising an amount of Rutin or its derivatives effective in activation of neuromedin U2 receptor to treat obesity and non-insulin dependent diabetes mellitus.

This invention provides a composition comprising an amount of Rutin or its derivatives effective in activation of neuromedin U2 receptor to decreases the level of total cholesterol, triglyceride, high-density lipoprotein cholesterol and lipoprotein cholesterol in blood.

This invention also provides a composition comprising an effective amount of a neuromedin U2 receptor agonist and a suitable carrier.

In an embodiment, the agonist is not previously known. This invention further provides this agonist. Regardless of whether the agonist is known or unknown, this invention provides a pharmaceutical composition comprising an effective amount of a neuromedium U2 receptor and a pharmaceutically acceptable carrier.

For the purposes of this invention “pharmaceutically acceptable carriers” means any of the standard pharmaceutical vehicles. Examples of suitable vehicles are well known in the art and may include, but not limited to, any of the standard pharmaceutical vehicles such as a phosphate buffered saline solutions, phosphate buffered saline containing Polysorb 80, water, emulsions such as oil/water emulsion, and various type of wetting agents.

This invention provides a method of treating insulin-dependent diabetes mellitus comprising administering to a diabetes mellitus subject or subject at risk of becoming diabetic an effective amount of Rutin or its derivative.

This invention provides a method for increasing the insulin sensitivity of a subject comprising administering to said subject an amount of Rutin or its derivative effective to increase the insulin sensitivity.

This invention provides a method for decreasing blood glucose of a subject comprising administering to the subject an amount of Rutin or its derivative effective to decreases the blood glucose level.

This invention provides a method for suppressing food intake and regulating energy homeostasis in a subject comprising administering to said subject an amount of Rutin or its derivative effective to suppress food intake and regulating energy.

This invention provides a method for reducing the body weight of a subject comprising administering to said subject an amount of Rutin or its derivative to reduce body weight.

This invention provides a method for treating obesity in a subject comprising administering to the said subject an effective amount of Rutin or its derivative.

This invention provides a method for decreasing total cholesterol, triglyceride, high-density lipoprotein cholesterol and lipoprotein cholesterol in blood of a subject comprising administering an effectively amount of Rutin or its derivative.

The above described subject includes human and other animals. In an embodiment, the subject is a mammal.

This invention provides the above method, the Rutin or its derivative is administered orally, intravenously or intraperitoneally. Other appropriate route may be also used as it will be adopted by a competent skilled artisan. Rutin or its derivative may be used with other agents.

As shown, infra, the dosage may be titrated used first in experimental animals then human. In an embodiment, the dosage is about 1 milligram (mg) to 1 gram (g) per kilogram of the subject used. In a further embodiment, it is about 1 milligram to about 100 milligram. In a still further embodiment, it is about 10 to 100 milligram per kilogram. In a still further embodiment, the dosage is about 10, 30 or 100 milligram respectively.

Other neuromedin U2 receptor agonists may be similarly used as described above.

This invention further provides a screening method for said agonist wherein the NMU-2R receptor and a reporter gene are introduced to a cell line in such a way that when an exogenous compound can activate the receptor which will lead to the reporter gene expression.

This invention provides a method for identifying neuromedin U2 receptor agonist comprising steps of obtaining cells which will activate a reporting system to produce a signal when contacting with an agonist of the neuromedin U2 receptor; contacting said cells with a compound; and detecting the signal from the reporting system of said cell where a positive signal indicates that the compound is an agonist. Various reporting systems are known in the art. In an embodiement, the system is a luciferase reporting system. In another embodiment, the system is a green fluorescent protein system.

As illustrated below in the Experimental Details section, a specific cell line is established. Stable transfected HEK-293 cells expressing human NMU-2R and a reporter gene were established for receptor agonist screening. Neuromedin U, a natural ligand for NMU-2R, activates the receptor and stimulates the expression of the reporter gene luciferase. The stable cell line was used to screen small molecule agonists of NMU-2R from a natural product compound library.

In this cell line, since NMU-2R is Gq coupled receptor, agonists will activate the receptor and increase IP3 production in the cell. The IP3 pathway will increase intracellular calcium concentration, and PKC pathway, and then activate transcription factors. The transcription factors will bind to MRE or SRE regions of the reporter gene construct and induce the reporter gene expression (Luciferase). Many other reporter genes may be used in this invention.

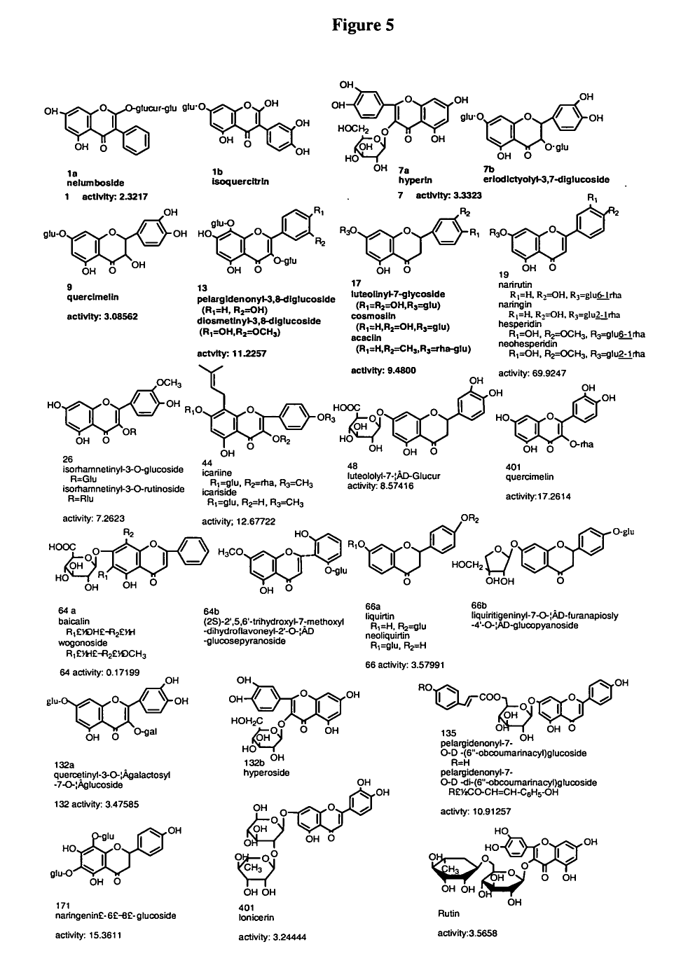

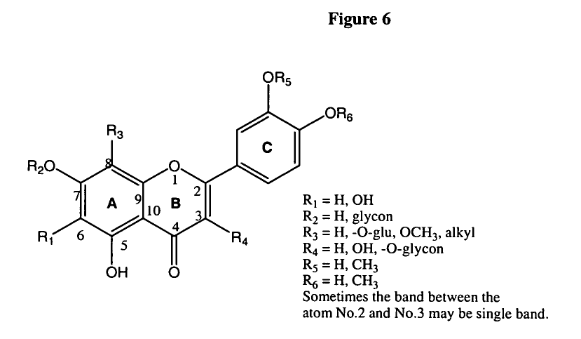

This invention also provides the agonist resulted from this screening method. In an embodiment, the agonist is depicted in FIG. 5. In another embodiment, the agonist has the base structure as set forth in FIG. 6.

This invention further provides the agonist which is not previously known. This invention also provides a pharmaceutical composition comprising an effective amount of the agonist and a pharmaceutically acceptable carrier. Like Rutin, this screened or identified agonist may be used similarly.

This invention will be better understood from the Experimental Details which follow. However, one skilled in the art will readily appreciate that the specific methods and results discussed are merely illustrative of the invention as described more fully in the claims which follow thereafter.

Experimental Details

Materials and Methods

Plasmids construction for NMU-2R and reporter gene: Human NMU-2R gene was cloned by PCR using cDNA from human hypothalamus as template. The two primers were 5′ ATG TCA GGG ATG GAA AAA CTT C and 5′ TCA GGT TTT GTT AAA GTG GAA GC. The PCR product was ligated into the pCR2.1-TOPO vector. The plasmid was digested with KpnI and XbaII and the DNA insert containing NMU-2R cDNA was subcloned into the mammalian expression vector pcDNA3.1(+). The identity of the human NMU-2R clone (NMU-2R/pCDNA3.1(+)) was confirmed by DNA sequencing.

Establishment of stable cell line: HEK-293 cells were maintained in DMEM with 10% FBS (Gibco) and 1% non-essential amino acid at 37° C. Cells were transfected with 2 μg of mixture of the reporter gene (3×MRE/CRE/SRE/Luci/pGL3^{[11]}) and NMU-2R/pCDNA3.1 in a ratio of 5:1 using Fugene 6 (Roche) according to the manufacturer's protocol. Twenty-four hours after transfection, cell culture medium was replaced with fresh medium containing G418 at final concentration of 800 mg/L. Stable cell line was generated using limited dilution based on the luciferase activity in the presence of NmU and forskolin (5 μmol/L). The stable cell line was named HEK-293/NMU-2R/3MCS/Luci cells.

Establishment of natural product library: Plants (Traditional Chinese Medicine) were collected from Southwestern area in China. Dried plant material (150 g) was ground to a homogenous powder. The powder was sonicated for 30 minutes in an organic solvent mixture of EtOH:EtOAc (50:50) followed by vigorous shaking for exhaustive extractions (two times, 4 and 8 hours each). After filtration and removing the organic solvents by rotary evaporation, the organic extract was obtained. Automated flash chromatography separations were performed on 50-gram Si flash columns (International Sorbent Technology Ltd., Mid Glamorgan, UK) using a Flash Master II automated chromatographic system (Jones Chromatography Inc., Lakewood, Colo.). Organic extract material (1 g) was dissolved in 5 mL MeOH:EtOAc (50:50) and adsorbed onto 5 g of silica powder. The dried powder was brought onto a 50 g silica column and eluted on the flash chromatography system using a step gradient of 1) 75% hexanes, 25% EtOAc, 2) 50% hexanes, 50% EtOAc, 3) 100% EtOAc, 4) 75% EtOAc, 25% MeOH, 5) 50% EtOAc, 50% MeOH. Preparative HPLC separations were performed on Betasil C18 columns (20×100 mm, Keystone Scientific Inc., Bellefonte, Pa., USA). HPLC system was assembled and consisted of Beckman System Gold 126 gradient HPLC pumps (Beckman Coulter Inc., Fullerton, Calif., USA). The system was controlled by Beckman 32 Karat chromatography software. Flash fraction materials (50 mg) were dissolved into 1 mL of MeOH:EtOAc (70:30). The materials were separated into 40 fractions (20 mL/min, 1 min per collection per tube) using the preparative HPLC system. The 40 tubes containing HPLC fractions were dried, dissolved into DMSO and transferred to 96-deep-well plates.

Luciferase assay and screening for NMU-2R agonists: The luciferase activity was measured according to the protocol from Promega. Briefly, 100 μL of HEK-293/NMU-2R/3MCS/Luci cells at 4×10⁴ cells/mL were added into each well of white 96 -well plate (flat, clear bottom, Costar). The cells were maintained in DMEM containing 10% FBS (Gibco) and 1% non-essential amino acid, and incubated at 37° C., 5% CO₂ overnight. Eleven μL of the mixture of forskolin (5 μmol/L) and the compounds (NmU or natural product compounds) were added into each well and incubated for 6-8 h. Finally, 110 μL of luciferase substrate Bright Glo™ (Promega) was added into each well. Luciferase activity was measured using Analysit HT (Molecular Device).

Structural identification of NMU-2R agonists: Two natural products, Epimedium and pericarpium citri reticulatae, have been analyzed by thin-layer chromatography (TLC) method based on the classic TLC protocols^{[12,13]}. The compounds from TLC plates were extracted using 1000 μL of methanol and their activities were detected using the reporter gene assay. Standard samples of the natural products with over 95% purity were purchased and used for the cell-based functional assay.

In vivo pharmacological studies: Rutin (Chengdu Plant Chemical Development Co. Ltd.) was dissolved in 1% of sodium carboxymethycellulose (CMC⁻Na⁺) and stored at 4° C. before use. Wistar rats and C57BL/6 mice (Shanghai SLAC Laboratory Animal Co. Ltd) were housed in an environment of 21±0.5° C. with a relative humidity of 50±10%. Every cage had a complete exchange of air 15-18 times per hour and a 12-h light-dark cycle with no twilight. Water and food were continuously available. Obese rats were developed using high fat diet for six weeks. Rutin was given to the animals at 6 mg/kg and 20 mg/kg (intraperitoneal injection) or 30 mg/kg and 100 mg/kg (oral) every 24 hours for 30 days. Body weights were measured two times a week for five weeks.

High fat diet induced obese rats were treated with Rutin at 100 mg/kg orally for 30 days and then fasted for 24 hours. Blood was taken from the animals and glucose, insulin, total cholesterol, high-density lipoprotein cholesterol and low-density lipoprotein cholesterol were measured. Blood glucose level was measured using Glucotrend (Roche) and insulin level was measured by radioimmunochemistry. Total cholesterol (TC), triglyceride (TG) and high-density lipoprotein cholesterol (HDLC) were measured using Beckman CX4 (Bechman). Low-density lipoprotein cholesterol (LDLC) was calculated by Friedewald formula (LDLC=TC−HDLC−0.456 TG). Glucose infusion rate was examined according to the standard protocol ^{[14]}. Briefly, insulin was infused into the venous cannula in the right lateral thigh at 1.67 mU.kg⁻¹.min⁻¹, and 10% of glucose solution was infused into the venous cannula in the left lateral thigh of anesthetized rats. Blood glucose level was measured every five minutes. The infusion rate of glucose was adjusted to make the blood glucose level at steady state. The glucose infusion rate was determined based on the average value of the infusion in one hour.

Experimental Results

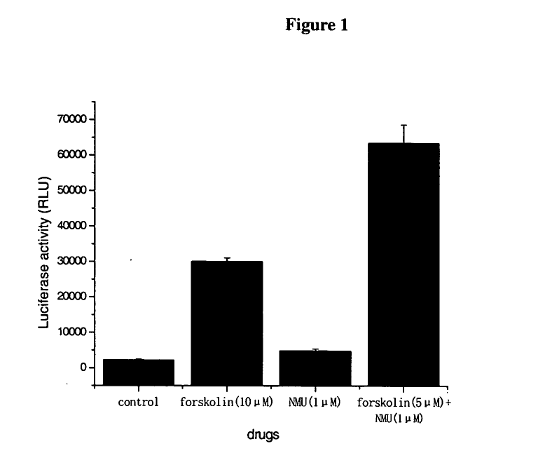

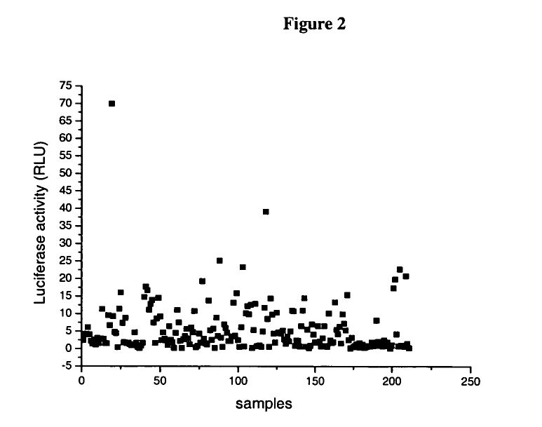

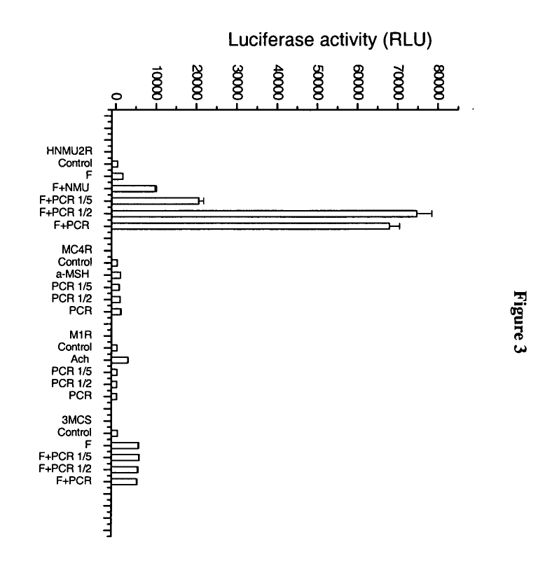

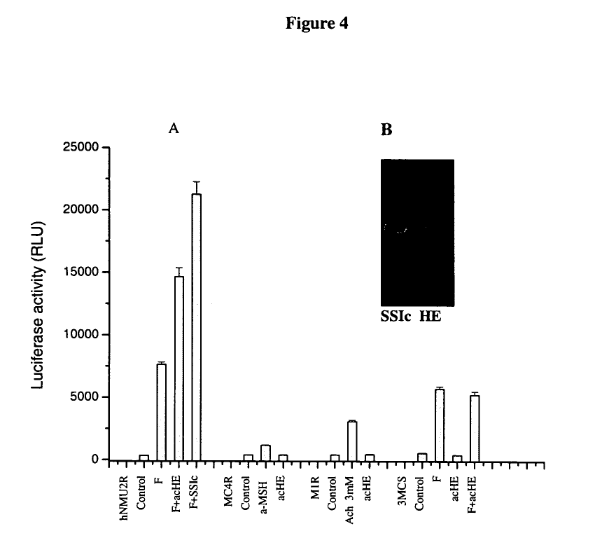

Identification of human NMU-2R agonists: Cell-based reporter gene assay was applied to screen natural compounds for NMU-2R receptor. It was found that the natural ligand NmU for NMU-2R gave the best response in the presence of 5 μmol/L forskolin (FIG. 1). Thus, the agonist screening was performed by adding compounds and forskolin to the stable cell line containing both NMU-2R and the reporter gene constructs. A number of natural product samples, such as compounds derived from Herba Epimedii and Pericarpium Citri Reticulatae, were identified as potential NMU-2R agonists from the proprietary natural product compound library (FIG. 2). Other receptors such as MC4 and M1 can't be activated by the two natural extracts (FIG. 3 and FIG. 4).

Since the natural product samples were mixtures of many compounds, it was further determined the effective compounds from the samples. One important feature of the proprietary natural product library is that all the samples have been derived from Traditional Chinese Medicine (TCM). In order to identify the effective compounds for NMU-2R, the compounds from Herba Epimedii and Pericarpium Citri Reticulatae were separated by using TLC. Icariin and Hesperidin, two purified flavonoid glycosides from Herba Epimedii and Pericarpium Citri Reticulatae respectively, were included in the TLC plates. Compounds were cut and extracted from the TLC and their activities were examined in the reporter gene assay. It was found that the active compounds were run at the same positions of Icariin and Hesperidin in the TLC plates, indicating these two flavonoid glycoside compounds were potential NMU-2R agonists (FIG. 4).

Structure and activity relationship analysis: To identify the essential core structure for NMU-2R agonist, the activities of different flavonoid glycosides from other TCM were systematically analyzed. 127 purified flavonoid glycoside compounds were collected and tested their activities in the NMU-2R reporter gene assay. In addition, a number of similar compounds, such as flavones and non-flavonoid glycosides, were examined in the reporter gene assay. The results demonstrated that most of flavonoid glycosides activated NMU-2R and led to the expression of the reporter gene luciferase (FIG. 5), while flavones and non-flavonoid-glycosides had no activity (Data not shown). Furthermore, the structure and activity relationship studies using 127 flavonoid glycoside compounds demonstrated that modifications on ring C of the core structure were essential for regulating the activity (FIG. 6). For example, —OH or —OCH₃ groups at ring C of flavonoid glycoside showed high activity on NMU-2R, while without these groups at ring C, such as flavonoid glycoside in Radix Scutellariae, had no effect on NMU-2R.

Several stable cell lines were generated to examine the specificity of the compounds. First, the activity of the compounds in cell line expressing the reporter gene alone were tested. Since the reporter gene cell line was not transfected with NMU-2R expression vector, compounds that can activate the reporter gene expression in this cell line were not NMU-2R agonists. In addition, the compounds in cell lines expressing other GPCRs were tested. More than twenty different GPCR stable cell lines were generated for the specificity assay. The results demonstrated that the flavonoid glycoside compounds had no effects on other GPCRs, suggesting that these compounds could be specific agonists for human NMU-2R.

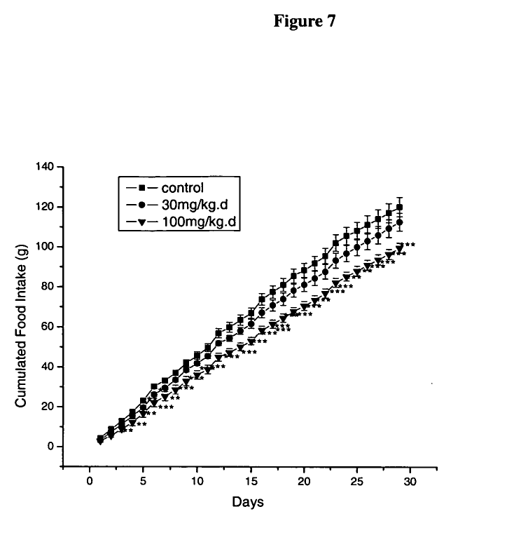

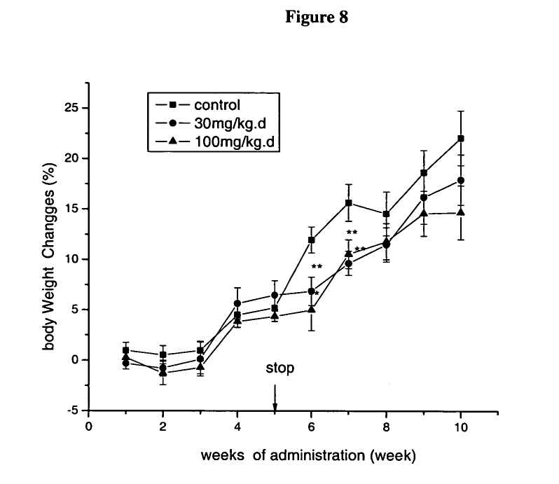

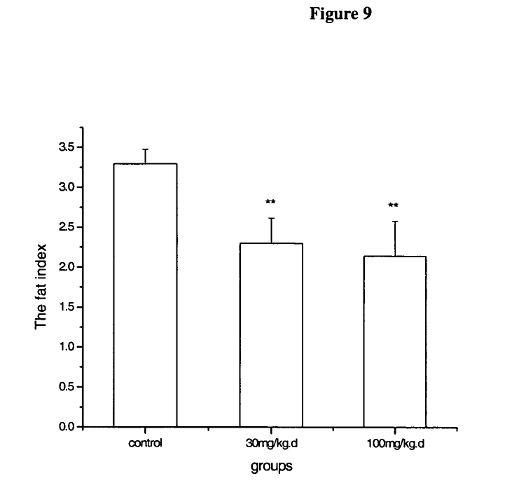

NMU2R agonist regulates body weight and low-density lipoprotein cholesterol (LDL-C): FIG. 4 showed the effects of Rutin on food intake in C57BL/6 mice. After 3 days of oral administration of Rutin at 100 mg/kg.d., the food intake of the animal started to show significant reduction (FIG. 7). There was no significant change in food intake after the administration of Rutin at 30 mg/kg.d. for 30 days. However, oral administration of Rutin at both 30 and 100 mg/kg.d. significantly reduced the bodyweight of the animals (FIG. 8). In addition, the Fat Indexes of these animals administrated with 30 and 100 mg/kg.d. were also decreased (FIG. 9). It is interesting to note that administration of Rutin at 30 mg/kg.d had no effect on food intake, but reduced both body weight and fat. These results suggested that NMU2R signal might regulate both food intake and energy expenditure.

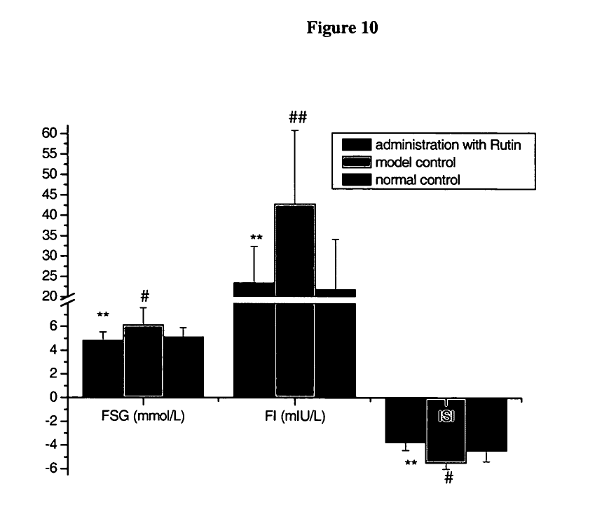

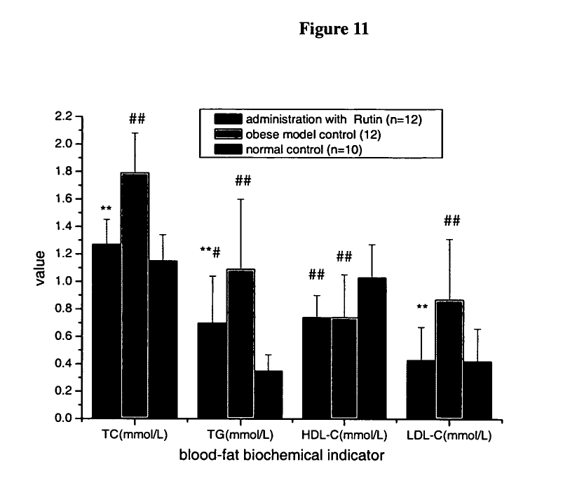

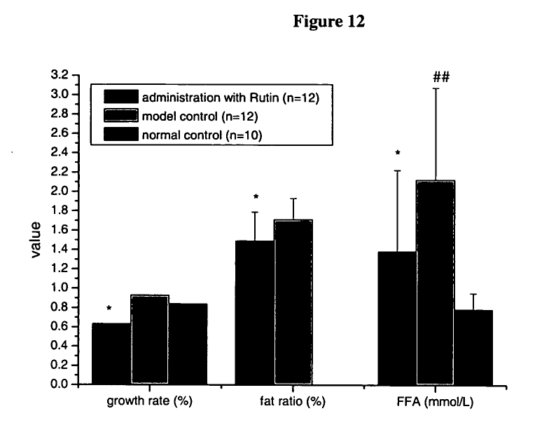

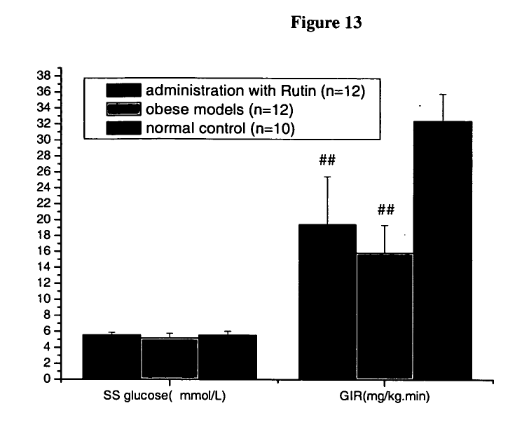

Improvement of insulin sensitivity and reduction of blood glucose level after administration of NMU2R agonists: Control and high fat diet induced Wistar rats were used for these experiments. The blood-fasting glucose, fasting insulin and insulin sensitivity were significantly increased in these animals (FIG. 10). After oral administration of 100 mg/kg Rutin for 4 weeks, the blood-fasting glucose and fasting insulin levels were significantly reduced (FIG. 10). The glucose infusion rate as a measurement for insulin resistance was examined. In the presence of 1.67 mU.kg⁻¹.min⁻¹ insulin infusion, glucose infusion rate was significantly reduced in obese rats compared to normal rats. This result demonstrated that high fat diet induced insulin resistance in the obese mice. Glucose infusion rate was significantly increase after Rutin treatment, indicating the drug prevented insulin resistance in the obese animal (FIG. 13). In addition, the effects of Rutin on blood TC, TG, LDL-C and HDL-C in high fat diet induced obese rats were examined. The results indicated that both blood TC and TG were elevated in obese animals. Surprisingly, although LDL-C was increased, HDL-C was decreased in the blood of obese rats. Rutin treatment significantly reduced TC, TG and LDL-C levels in the obese mice, while HDL-C level remained the same as control animals (FIG. 11). Measurements were taken of the ratio of fat to body weight as well as blood fatty acids concentration after Rutin treatment. These results demonstrated Rutin effectively decreased the fat ratio and reduced the concentration of fatty acids in the blood of obese rats (FIG. 12).

Experimental Discussion

Reporter gene assay for human MNU-2R agonists screening: Human NMU-2R is a G-protein coupled receptor mediating intracellular Ca²⁺ signaling. Natural ligand NmU activates the receptor with potency in nanomolar range^{[5-7,15-20]}. Pharmacological studies demonstrated that NMU-2R activated phospholipase C (PLC) and generated inositol 1,4,5-trisphosphate^{[21]}. Furthermore, activation of human NMU-2R in CHO cells and HEK-293 cells resulted in inhibition of forskolin-stimulated cAMP accumulation [16, 21] Immunoprecipitation of specific G-protein α-subunits from cell membranes in the presence of [³⁵S]-GTPγS have shown the interaction of human NMU-2R receptor with both Gαq/11 and Gαi^{[21]}. Based on these pharmacological properties of the receptor, a cell-based reporter gene assay to screen the human NMU-2R agonists was generated. The reporter gene contains 3×MRE, SRE, and CRE response elements from mini VIP promoter followed by the reporter gene luciferase. A stable HEK-293 cell line expressing both human NMU-2R and the reporter gene construct was generated. It is interesting to note that the natural ligand NmU only slightly induced the reporter gene expression, but combination of NmU and forskolin significantly increased luciferase expression. This result supported Brighton's suggestion that human NMU2R receptor coupled with both Gαq/11 and Gαi^{[21]}. Stimulation of human NMU-2R could activate Gαq/11 and increase the intracellular concentration of calcium, thereby activate MRE and induce the expression of reporter gene. At the same time, activation of the receptor coupled with Gαi, resulted in the inhibition of adenylate cyclase (AC) and decreased the reporter gene expression. Forskolin treatment could activate AC and block the inhibitary effect of Gαi. This activity could increase the intracellular cAMP concentration and activate CRE, therefore, induce the reporter gene expression. Taken together, the reporter gene assay system has been successfully applied for NMU-2R agonists screening. Using the reporter gene assay, small molecule agonists for human NMU-2R were identified. The agonists are flavonoid glycosides and thire structures are summarized in FIG. 8. It was found that non-flavonoid glycosides did not show any activity. In addition, flavones could not activate the receptor, suggesting that the glycon groups played an important role in biding or activating the receptor. Structure and activity relationship analysis demonstrated that R₂ of the flavonoid glycoside was essential for the agonist activity. Detailed structure analysis demonstrated that without —OH or —OCH₃ on C ring, the compounds could not activate the receptor, suggesting that the —OH or —OCH₃ group on C ring of the flanonoid glycosides may participate in binding to the receptor.

Involvement of NMU-2R in regulation of body weight, blood LDL-C, insulin sensitivity and blood glucose: Much evidence indicated that NmU played important roles in regulating food intake and body weight. Intracerebroventricular injection of NmU decreases food intake and body weight in rodents^{[5,17,22-25]}. Furthermore, intracerebroventricular injection of NmU antiserum increased food intake in rats^{[17]}. Gene expression analysis demonstrated that fasting reduced levels of NmU in the ventromedial hypothalamus^{[5]}. The effect of NmU has been mapped to particular hypothalamic nuclei. Injection of NmU into either the PVN or arcuate nucleus immediately decreases food intake in rats^{[25]}. Recently, NmU gene knockout in mice further confirmed the activity of the peptide ligand could regulate body weight. Furthermore, the mutant mice showed hyperglosimia and hyperinsulinemia phenotype, suggesting NmU also play important roles in regulating blood glucose and insulin sensitivity^{[26]}.

Although much evidence demonstrated that NmU regulated food intake, body weight, and insulin sensitivity, it was not clear which NmU receptor participated in the activity. It has been shown that NMU-2R was expressed predominantly in the central nervous system and in particular has been localized to the PVN and arcuate nucleus of hypothalamus in rat and mouse^{[5,9]}, suggesting that the NmU activity was mediated via NMU-2R receptor.

Chemical genetic approach has been taken to investigate the function of NMU-2R. Rutin is one of the flavonoid glycosides that can activate NMU-2R in a cell-based functional assay.

The compound had no activity for NMU-1R and other G-protein coupled receptors, indicating it was a NMU-2R specific agonist. The administration of Rutin in femal C57BL/6 mice and obese rats resulted in a significant inhibition of feeding and reduction of body weight. The studies also indicated that Rutin could reduce blood-fat level, blood-glucose level, and improve insulin sensitivity. These results strongly suggested that the anti-obesity, reduction of blood-fat and glucose level, as well as improvement of insulin sensitivity effects of NmU was regulated through NMU-2R.

The molecular mechanism and signal pathways that hNM-2R agonists regulate feeding behavior and energy balance are unclear. One possibility of the agonist effects is through corticotrophic releasing hormone (CRH) signal pathway. It has been shown that intracerebroventricular administration of CRH could also inhibit feeding in rats^{[27]}. The NmU effects on suppression of food intake, elevation of oxygen consumption, and regulation of body weight were completely eliminated in CRH knockout mice^{[28]}. CRH containing neurons have been found in PVN region of hypothalamus. In addition, NmU could stimulate the release of CRH from hypothalamus^{[8]}. These data strongly suggested that activation of NMU-2R may increase release of CRH and regulate feeding behavior as well as energy homeostasis. Futher studies, such as phenotypic analysis of NMU-2R knockout or NMU-2R/CRH double knockout mice, may help to fully understand the molecular basis and physiological function of the receptor.

Rutin and its derivative are the first reported non-peptide agonist for NMU-2R. These results suggested that activating NMU-2R can suppress food intake and regulate of body weight. Pharmacological characterization of the NMU-2R agonists could be the first and essential step for the development of anti-obesity and type II diabetes drugs.

### REFERENCES

1. Brighton, P. J., P. G. Szekeres, and G. B. Willars, Neuromedin U and its receptors: structure, function, and physiological roles. Pharmacol Rev, 2004. 56(2): p. 231-48.
2. Bishop, A. E., et al., The distribution and development of a new brain and gut peptide, neuromedin-U. J Pathol, 1988. 154: p. A99-A100.
3. Augood, S. J., J. R. Keast, and P. C. Emson, Distribution and characterisation of neuromedin U-like immunoreactivity in rat brain and intestine and in guinea pig intestine. Regul Pept, 1988. 20(4): p. 281-92.
4. Honzawa, M., et al., Neuromedin U-like immunoreactivity in rat intestine: regional distribution and immunohistochemical study. Neuropeptides, 1990. 15(1): p. 1-9.
5. Howard, A. D., et al., Identification of receptors for neuromedin U and its role in feeding. Nature, 2000. 406(6791): p. 70-4.
6. Hedrick, J. A., et al., Identification of a human gastrointestinal tract and immune system receptor for the peptide neuromedin U. Mol Pharmacol, 2000. 58(4): p. 870-5.
7. Raddatz, R., et al., Identification and Characterization of Two Neuromedin U Receptors Differentially Expressed in Peripheral Tissues and the Central Nervous System. J Biol Chem, 2000. 275(42): p. 32452-9.
8. Wren, A. M., et al., Hypothalamic Actions of Neuromedin U. Endocrinology, 2002. 143(11): p. 4227-34.
9. Graham, E. S., et al., Neuromedin U and Neuromedin U receptor-2 expression in the mouse and rat hypothalamus: effects of nutritional status. J Neurochem, 2003. 87(5): p. 1165-73.
10. Rover, S., et al., High-affinity, non-peptide agonists for the ORL1 (orphanin FQ/nociceptin) receptor. J Med Chem, 2000. 43(7): p. 1329-38.
11. Jiang, C., et al., Generation of a bioactive neuropeptide in a cell-free system. Anal Biochem, 2003. 316(1): p. 34-40.
12. China, P.b.o.t.P.s.R.o., Pharmacopoeia of the People's Republic of China: kromogram collection of thin-layer chromatography of traditional Chinese medicine., ed. 6. 1993: GungDong technology publishing company. 53-54.
13. China, P.b.o.t.P.s.R.o., Pharmacopoeia of the People's Republic of China: kromogram collection of thin-layer chromatography of traditional Chinese medicine., ed. 6. 1993: GungDong technology publishing company. 72.
14. Ding, S. Y., Z. F. Shen, and M. Z. Xie, Preliminary study of insulin resistance induced by neonatal monosodium glutamate treatment in normal wistar rats. chinese pharmacological bulletin, 2oo1. 17(2): p. 81-5.
15. Fujii, R., et al., Identification of neuromedin U as the cognate ligand of the orphan G protein-coupled receptor FM-3. J Biol Chem, 2000. 275(28): p. 21068-74.
16. Hosoya, M., et al., Identification and functional characterization of a novel subtype of neuromedin U receptor. J Biol Chem, 2000. 275(38): p. 29528-32.
17. Kojima, M., et al., Purification and identification of neuromedin U as an endogenous ligand for an orphan receptor GPR66 (FM3). Biochem Biophys Res Commun, 2000. 276(2): p. 435-8.
18. Shan, L., et al., Identification of a novel neuromedin U receptor subtype expressed in the central nervous system. J Biol Chem, 2000. 275(50): p. 39482-6.
19. Szekeres, P. G., et al., Neuromedin U is a potent agonist at the orphan G protein-coupled receptor FM3. J Biol Chem, 2000. 275(27): p. 20247-50.
20. Funes, S., et al., Cloning and characterization of murine neuromedin U receptors. Peptides, 2002. 23(9): p. 1607-15.
21. Brighton, P. J., et al., Signaling and ligand binding by recombinant neuromedin U receptors: evidence for dual coupling to Galphaq/11 and Galphai and an irreversible ligand-receptor interaction. Mol Pharmacol, 2004. 66(6): p. 1544-56.
22. Nakazato, M., et al., Central effects of neuromedin U in the regulation of energy homeostasis. Biochem Biophys Res Commun, 2000. 277(1): p. 191-4.
23. Niimi, M., K. Murao, and T. Taminato, Central administration of neuromedin U activates neurons in ventrobasal hypothalamus and brainstem. Endocrine, 2001. 16(3): p. 201-6.
24. Ivanov, T. R., et al., Evaluation of neuromedin U actions in energy homeostasis and pituitary function. Endocrinology, 2002. 143(10): p. 3813-21.
25. Hanada, R., et al., A role for neuromedin U in stress response. Biochem Biophys Res Commun, 2001. 289(1): p. 225-8.
26. Hanada, R., et al., Neuromedin U has a novel anorexigenic effect independent of the leptin signaling pathway. Nat Med, 2004. 10(10): p. 1067-73.
27. Morley, J. E. and A. S. Levine, Corticotrophin releasing factor, grooming and ingestive behavior. Life Sci, 1982. 31(14): p. 1459-64.
28. Hanada, T., et al., Central actions of neuromedin U via corticotropin-releasing hormone. Biochem Biophys Res Commun, 2003. 311(4): p. 954-8.

1. A composition comprising an amount of a neuromedin U2 receptor agonist or its derivative effective in activation of neuromedin U2 receptor.

2. A composition of claim 1 wherein neuromedin U2 receptor agonist is isolated from a natural product library.

3. A composition comprising an amount of neuromedin U2 receptor agonist or its derivative effective in activation of neuromedin U2 receptor to increase the insulin sensitivity.

4-17. (canceled)

18. A method for identifying neuromedin U2 receptor agonist comprising steps of:
a. Obtaining cells which will activate a reporting system to produce a signal when contacting with an agonist of the neuromedin U2 receptor;
b. Contacting said cells with a compound; and
c. Detecting the signal from the reporting system of said cell where a positive signal indicates that the compound is an agonist.

19. The compound identified by the method of claim 18 which is not previously known.

20. A composition comprising an effective amount of the compound identified by the method of claim 18 and a suitable carrier.

21. Uses of the compound identified by claim 18 or other neuromedin U2 receptor agonist for treating insulin-dependent diabetes, increasing insulin sensitivity, decreasing blood glucose, suppressing food intake and regulating energy homeostatis, reducing body weight, treating obesity, and decreasing total cholesterol, triglyceride, high-density lipoprotein cholesterol and lipoprotein cholesterol.
